(19) United States
(12) Patent Application Publication
(10) Pub. No.: US 2002/0133067 A1
(43) Pub. Date: Sep. 19, 2002

(54) New born and premature infant SIDS warning device

(76) Inventor: William H. Jackson III, Wilmington, NC (US)

Correspondence Address: Michael E. Mauney, Attorney at Law, P.O. 10266, Southport, NC, 28461, US

(21) Appl. No.: 09/808,978

(22) Filed: Mar. 15, 2001

(51) Int. Cl. A61B 5/00

(52) U.S. Cl. 600/323; 600/344; 600/310

(57) ABSTRACT

A device for use with very small newly born or premature infants to monitor and prevent the onset of a Sudden Infant Death Syndrome event. A pulse oximeter is mounted in an opaque foot wrap, which is wrapped around the infant's foot in the area of the arch and ball of the foot The foot wrap is connected to a second wrap, which is wrapped around the ankle. The ankle wrap secures the foot wrap portion from rotational motion or from sliding forward toward the toes on an infant's foot. Blood oxygen and pulse read-outs on the pulse oximeter are transmitted to a monitor kept by the caregiver. Visible read-outs for the blood oxygen or pulse are shown on the monitor. The monitor sounds an alarm if the infant's blood oxygen drops to a predetermined level for a predetermined time. When not in use, the device is recharged on a stand.

### BACKGROUND OF THE INVENTION

1. Field of the Invention

This invention relates to a home monitoring device to be used to detect warning signs of Sudden Infant Death Syndrome (SIDS) and other respiratory or cardiac conditions threatening to premature, newborn, or very small infants. This invention is designed to minimize false alarms, to be used by caregivers who have no specialized training, to be portable, affordable, and practical.

2. Related Patents

This invention grows out of the work that led to my U.S. Pat. No. 6,047,201 that issued on Apr. 4, 2000. That patent is incorporated by reference herein.

3. Description of Related Art

Sudden Infant Death Syndrome (SIDS) is a leading cause of infant death. The cause of SIDS is unknown. Infants who have periods of apnea, changes in skin color, changes in muscle tone, or who may require help in breathing are more likely to die of SIDS. It has been thought that an infant's sleeping position can be a factor in development of SIDS and various arrangements of pillows around the infant have been proposed to reduce the risk of SIDS.

If a caregiver or parent has sufficient warning of the development of a SIDS episode in an infant, than caregiver may be able to intervene to prevent the infant death. One way of detecting an onset of SIDS is if the infant has stopped breathing or if the heart rate drops significantly. Apnea/bradycardia alarm systems have been available for home use since the late 1970's. These alarm systems attach electrodes to an infant's skin. These alarm systems sound an alarm should an infant stop breathing or should an infant's heart rate drops below a preset level. These alarm systems have proved impractical because they require electrodes attach to an infant's skin, which are connected to a monitor by wires. These wires can be uncomfortable or can even be hazardous to an infant. Moreover, in practice it has been found these monitoring systems give many false alarms. Each false alarm both causes anxiety and can lead to reduced watchfulness by caregivers over time. Therefore, for a SIDS monitoring system to be practical and successful, false alarms must be minimized.

Pulse oximeters have also been employed to monitor for the beginning of a SIDS episode. A pulse oximeter measures the level of oxygen saturation in the blood. If the blood oxygen drops to an unsafe level (hypoxemia), this is a sign of a beginning episode of SIDS. Pulse oximeters operate by measuring a light signal passed through a portion of the body. As the oxygen saturation of the red blood cells changes, it affects the light signal hence, can be used to measure the oxygen saturation in the red blood cells. Pulse oximeters are typically placed on a finger, hand, toe, or foot of an infant being monitored. It is possible to place an oxygen saturation probe on an infant's sternum or back, reducing the motion of the probe. Motion can cause false alarms. Thus, it is desirable to reduce the motion of the probe. However, probes placed on an infant's sternum or back are usually connected by wires to central monitoring units. These wires have drawbacks. They can be uncomfortable in use or even dangerous if tangled.

One system has been proposed that uses a combination of pulse oximeter, motion detector, and video camera (see Kim, U.S. Pat. No. 5,505,199). This proposes a motion sensor, video camera and pulse oximeter all be placed in the infant's room and connected to a central unit in the monitoring room, usually the parent's room. The Kim '199 patent states claims that the use of a motion detector with a pulse oximeter and with a video monitor is successful in reducing the number of false alarms. However, it requires a video camera and a motion detector and a pulse oximeter all connected to a video monitor. This requires extensive wiring and instrumentation and is prohibitively expensive for most parents. Additionally, it presumes the monitoring will only occur from the location of the central monitoring unit, including the video display. This means that the caregiver must be close to the central monitoring unit while the device is in use. Also the infant must be sleeping in the room equipped with the camera, motion detector, and oximeter.

Pulse oximeters may use a sensor attached to a finger or other extremity. This sensor is conductively coupled to an electronic device that measures and provides a readout of the percentage of oxygen in arterial blood. For example, the Isaacson et al., U.S. Pat. No. 5,490,523 discloses a pulse oximeter with a miniature measuring readout device attached and incorporating the sensor. The Isaacson design eliminates the conductive cables that connect the sensor to the readout. Isaacson claims the conductive cables can become damaged during use. His design eliminates the connecting cable, hence, eliminates the problem of cable connection failure. The Isaacson et al. patent shows an extremely small, lightweight and durable pulse oximeter that is battery operated.

Halleck et al., U.S. Pat. No. 5,549,113, suggests a dual frequency transmitter for remote monitoring of selected physiological parameters. The dual frequency assures against a loss of one signal transmission, hence against a loss of data. In Halleck's best mode, the detectors used are for respiration and electrocardiogram readings. Halleck suggests that these should be directly mounted on a subject including extremities.

Athan et al., U.S. Pat. No. 5,575,284, discloses a compact, portable pulse oximeter utilizing two light emitting diodes. This pulse oximeter uses a light to a frequency converter and a high-speed counter to remedy perceived deficiencies in prior art. Athan et al. suggests that their version of a pulse oximeter can be made small enough and light enough to be worn by an ambulatory patient, like a wrist watch, bracelet, anklet, inflatable cuff, and so on might be worn.

Infants are sometimes monitored by use of a sound receiving and transmit unit called a baby monitor. Here, a broadcast unit incorporates a microphone and a radio transmitter. This is placed in proximity to an infant. Should the infant cry or make unusual noises, the microphone will pick up the sound, which will then be transmitted by the radio transmitter to a receiving unit with a receiver and a speaker. Thus, a parent or caregiver in a remote room can hear an infant cry by use of the baby monitor. The baby monitor is compact enough so that a parent or caregiver may carry it with him or her as they move about the house. However, this is not effective for monitoring an infant in a SIDS episode. It is believed that SIDS is a silent event, hence not signaled by any sound made by an infant.

It is believed that none of the above described devices provide an effective way for parents or caregivers to monitor an infant to detect a SIDS event. For that reason, I designed a compact battery-powered pulse oximeter unit using a small transmitter unit to communicate appropriate data gathered by the pulse oximeter to a remote monitoring unit (U.S. Pat. No. 6,047,201). A monitoring unit would be used by a parent or caregiver, much like a receiver or a baby monitor, to monitor an infant's blood oxygen hence, to receive a early warning of the onset of a SIDS episode. This invention used a toe cap to house the pulse oximeter. However, in very small infants, including premature infants or the newly born, it is difficult to place and secure a toe cap in place on their very small great toe. Additionally, a toe cap, especially on a small infant, can be subject to false readings due to signal artifacts. Ordinarily, signal artifacts have three major sources. The first is ambient light. That is, the light that is used to measure the oxygen content in the blood can be masked or otherwise distorted by outside light This is especially a problem where the tissue itself is thin and translucent. For example, if the toe cap is not securely placed around the toe of a very small infant, the ambient light interferes with the pulse oximeter, resulting in false readings. Second, a signal artifact may be caused by low perfusion of the blood in the extremity being measured. Consequently, pulse oximeters placed on extremities such as toes or fingers, which may be subject to poor circulation for a variety of reasons, can result in inaccurate readings. The third is for the patient or the sensor motion. For example, sensor motion can be a problem in a tow cap secured to the very small toe of a premature or newly born infant. If the toe cap is secured too tightly, it can affect the perfusion. If the toe cap is secured too loosely, the sensor may move or slide around the toe. Consequently, it is believed, while a toe cap is adequate for many applications, it is not preferable in applications for very small infants, especially the newborn or the premature.

### SUMMARY OF THE INVENTION

It would be desirable to have a pulse oximeter which may be securely mounted to the foot of a very small infant, especially the newborn or a premature infant. The pulse oximeter should be mounted in such a way as to completely eliminate the possibility of ambient outside light causing signal artifacts or inaccurate readings. It should be mounted in such a way as to be secured to the foot of the infant to preclude as much as possible the motion of the sensor but at the same time having little, if any, impact on the perfusion of the blood vessels which are supplying the blood being tested by the pulse oximeter through light transmittance. The device should be light, compact, easy to apply by an untrained caregiver, reliable, practical, and inexpensive enough to be widely used.

The current invention has a pulse oximeter mounted in a woven foot wrap. The wrapping material is made of an opaque elastic material. The wrap will secure not only around the arch and ball area of the infant's foot, but also around the ankle. The pulse oximeter will be placed in the wrap on the dorsal and plantar area near the arch of the infant's foot. The pulse oximeter will be powered by a battery. It will use a low-powered transmitter to transmit readings to a remote monitoring unit. The remote monitoring unit will also be battery-powered, portable, and to be used by a caregiver to constantly monitor the transmitted data signals to guard against the onset of a SIDS episode in an infant using the device.

### BRIEF DESCRIPTION OF THE DRAWINGS

FIG. 1 shows the foot sensing unit of the current invention seen from the side in place on an infant's foot

FIG. 2 shows the foot sensing unit of the current invention unfolded and seen from above.

FIG. 3 shows the monitoring unit with a portion shown in cut-a-way.

FIG. 4 shows the monitoring unit with a different portion shown in cut-a-way.

FIG. 5 shows a diagram of the foot sensing portion of the current invention.

FIG. 6 shows a diagram of the monitoring unit.

FIG. 7 shows a flow chart for the controller logic employed by the invention.

FIG. 8 shows the current invention in use.

FIG. 9 shows the current invention being recharged.

### DETAILED DESCRIPTION OF THE DRAWINGS

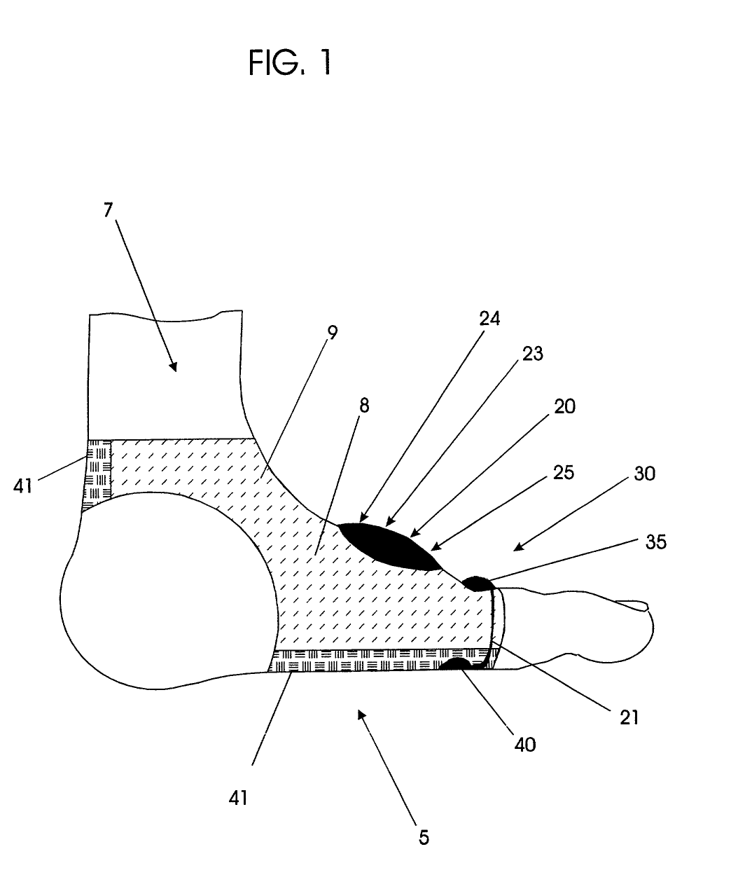

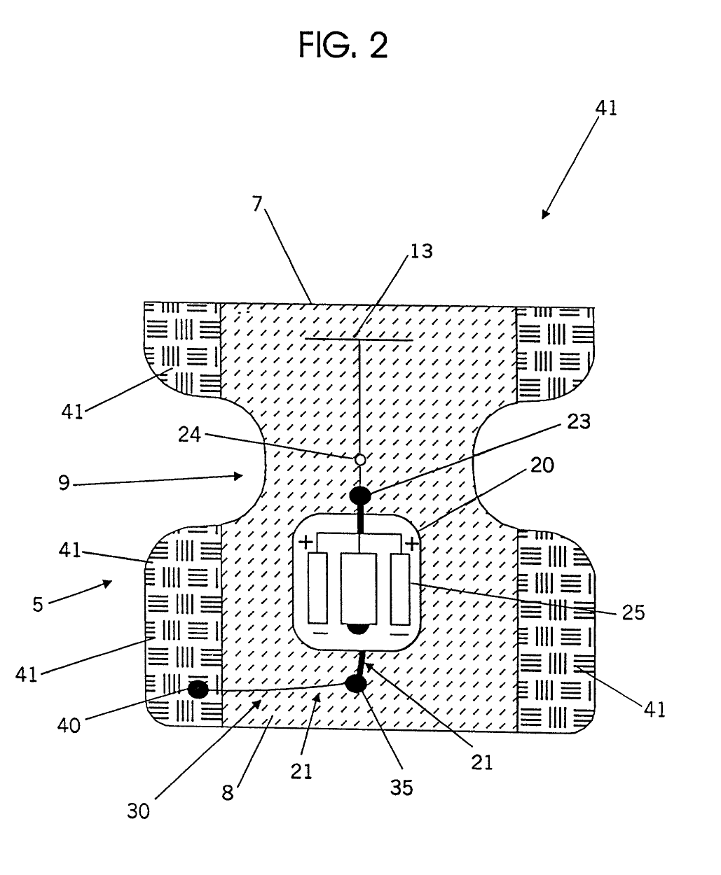

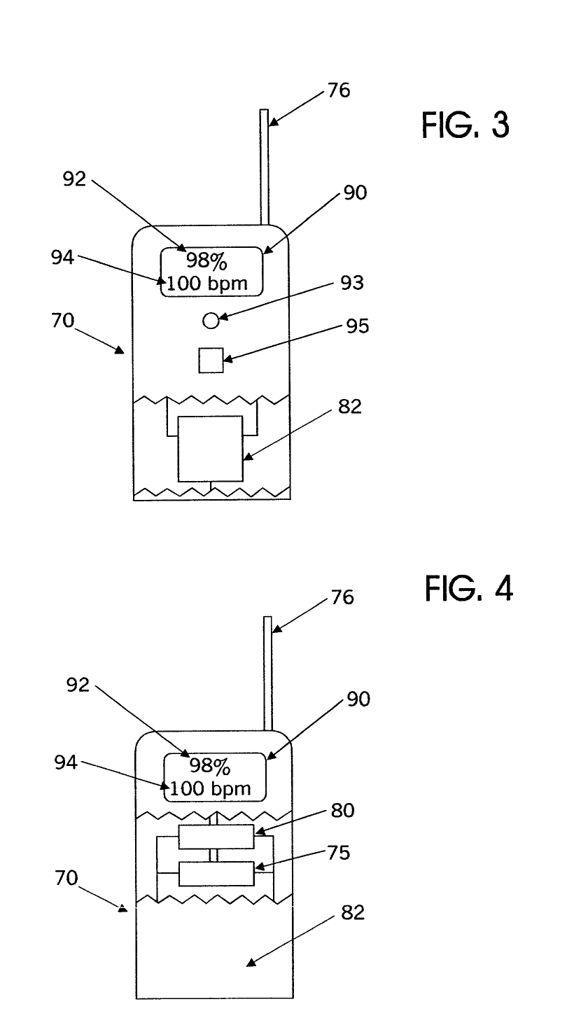

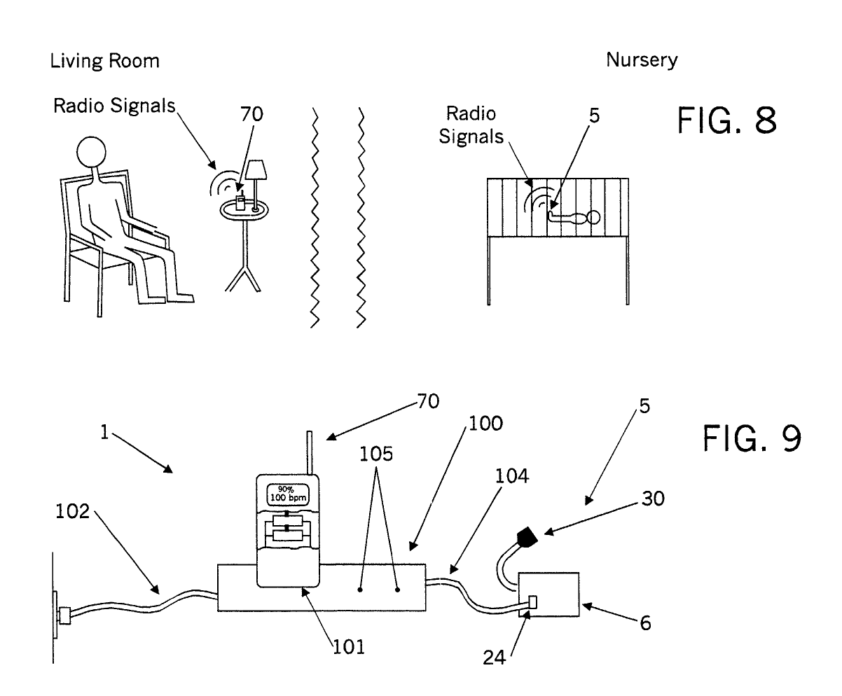

The infant SIDS warning device (1) (seen in FIG. 8) has two operating parts. First is the foot sensing unit (5) shown in FIGS. 1 and 2. Second is the monitoring unit (70) shown in FIGS. 3 and 4. First consider FIG. 1. The foot sensing unit (5) of the current invention is shown wrapped around an infant's foot. The foot sensing unit (5) has an ankle wrap portion (7) and an arch wrap portion (8) connected to each other by a connecting portion (9). In the arch wrap portion (8) there is a transmitting unit (20) mounted within the arch wrap portion (8) on the top or dorsal aspect of the foot. The transmitting unit (20) is placed within the arch wrap portion (8) so that when the arch wrap portion (8) is wrapped around an infant's foot, the transmitting unit (20) will be on the top of the infant's foot approximately above the arch of the infant's foot. The transmitting unit (20) requires a power source (25) for powering the transmission unit and also powering the oximeter (30). As part of the power source (25) there is a low-voltage sensor (not shown) connected to a warning light (23). This is to advise users in the event the power source (25) has become depleted. For most applications, the power source (25) will consist of batteries of conventional design. Replaceable mercury or longer lasting replaceable lithium batteries can be used. However, the preferred power source (25) is a rechargeable nickel cadmium battery. If the rechargeable nickel cadmium batteries is used as the power source (25), a charging unit (24) will be in proximity to the power source (25). The oximeter (30) contains a light emitting unit (35) and a light sensing unit (40) connected by wire (21) to the power source (25). Light is emitted by the light emitting unit (35) and passes from the dorsal area to the plantar area of the infant's foot where it is seen by the light sensing unit (40). This area of the foot is well supplied with arterial blood. Placement of the pulse oximeter (30) in the arch and/or ball area of the foot help prevent artifacts arising from ambient light. For example, ambient light can easily penetrate a toe cap, especially one that is attached to the great toe of a very small infant such as a newly born or premature infant. Secondly, an extremity like a finger or toe is more likely to experience perfusion problems than the ball and arch area of the foot. Finally, if the light emitting unit (35) and the light sensing unit (40) slide or move in their place of mounting, it can cause artifactual readings. The only way of securing a toe cap in place is to apply enough pressure around the toe to keep the light emitting unit (35) and the light sensing unit (40) in place. This can restrict circulation of blood in the toe causing perfusion problems, hence artifactual readings. However, here the arch wrap portion (8) of the foot sensing unit (5) is substantially larger than a toe cap and can be secured over a much wider area providing for a better frictional fit requiring less pressure to keep in place. Moreover, the ankle wrap portion (7) of the foot sensing unit (5) may be securely wrapped around the ankle to serve as a double anchoring point in a different plane. This helps to prevent motion of the pulse oximeter (30). The ankle wrap portion (7) makes it impossible for the foot sensing unit (5) to slide laterally around the foot. The ankle wrap portion (7) also prevents the foot sensing unit (5) from sliding forward toward the toes of the foot. Because the foot increases in size, moving from the toe to the ankle portion of the foot, if one wraps the arch wrap portion (8) carefully, and securing it in place using miniature hook-and-eye securing means (41) such as VELCRO, then the arch wrap portion (8) will not slide toward the ankle. The foot sensing unit (5) may be very securely fixed in place, but without requiring such pressure as to affect the perfusion of blood that may induce artifactual readings due to low perfusion.

The general construction and functioning of pulse oximeters are well known to those of skill in the art. Among manufacturers of pulse oximeters are Palco Labs, Medical Systems International Corporation, Promedix, among many others. Usually a pulse oximeter uses a light emitting diode to emit light at different frequencies. The light passes through the portion of a body on which the pulse oximeter is placed. A light sensor is placed to receive the light after it is passed through that portion of the body to which the sensor is applied. Commonly, the sensor is immediately opposite the light emitting diode, as is shown in FIG. 1. However, it may be adjacent to the light emitting diode in reflective pulse oximeters where a reflecting piece is placed opposite from the light emitting diode so the light first passes through a portion of the body of a patient, is reflected by the reflective piece to pass back through that portion of the body of the patient to be received by the sensors. Because the light must successfully pass through the body for a pulse oximeter to work, it is usually applied to an extremity of a patient thin enough to allow the light to pass through. Common areas of placement for a pulse oximeter are a finger, toe, bridge of the nose, or the ear. However, in an infant the foot may also be used, as is shown in FIG. 1.

In FIG. 2, the foot sensing unit (5) is unwrapped from the foot of the infant, laid out flat, and viewed from above. The ankle wrap portion (7) is seen at the top of FIG. 2. At opposing ends of the ankle wrap portion (7) are miniature hook-and-eye connecting means (41) commonly known by the trade name VELCRO. The hook portions from the viewer's perspective is at the right wing of the ankle wrap portion (7), while the eye portion would be underneath and out of sight from a view from above on the left side of the ankle wrap portion (7). However, here it is shown as a series of small lines. Ordinarily, the foot sensing unit (5) will be made, at least in part, of a breathable, opaque, somewhat elastic material, not unlike materials used in wrap bandages for ankle support. The fit of the foot sensing unit (5) is very important for the overall functioning of the unit. The ankle wrap portion (7) will fit securely and snugly around the ankle of an infant so it will not be dislodged by kicking movements common in small infants. To that end, a woven elastic material provides for a snug fit, but not so snug as to affect blood circulation of the foot. The elastic material also provides some degree of adjustability. Adjustability is also provided by the hook-and-eye connecting means (41). The hook-and-eye connecting means (41) need not mate exactly in order to provide secure attachment to the ankle wrap portion (7) around the ankle of an infant. The arch wrap portion (8) is designed similarly to the ankle wrap portion (7). At least a portion of the arch wrap portion (8) will be made of a stretchable, woven, opaque material for a comfortable but secure fit Hook-and-eye connecting means (41) will be placed at the ends of the wings of the arch wrap portion (8), as was described for the ankle wrap portion (7). As with the ankle wrap portion (7), the hook-and-eye connecting means (41) on the arch wrap portion (8) need not mate exactly to provide a secure and tight fit. The transmitting unit (20) is seen centered between the two hook-and-eye connecting means (41) on the arch wrap portion (8). Flanking the transmitting unit (20) are two batteries shown as the power source (25). The batteries provide power for the transmitting unit (20), as well as the pulse oximeter (30). The light emitting unit (35) is seen placed just below the transmitting unit (20) and is connected by wire (21) to the power source (25). The light receiving unit (40) is spaced apart from the light emitting unit (35) so that when the arch wrap portion (8) is wrapped into place, the light receiving unit (40) will be opposite from the light emitting unit (35) on the foot of an infant in operation. The light sensing unit (40) is placed in a wing of the arch wrap portion (8), here seen in the left wing, so it will fold into place on the plantar area of the foot of an infant, so that the light sensing unit (40) may be placed opposite from the light emitting unit (35) on the top of the foot. The right wing of the arch wrap portion (8) of the light emitting unit (35) will be folded into place over the light sensing unit (40) where the hook-and-eye connecting means (41) will mate to hold the light sensing unit (40) and light emitting unit (35) in the correct position. The ankle wrap portion (7) will be securely wrapped around the ankle of the patient The connecting portion (9) secures the ankle wrap portion (7) and the arch wrap portion (8) in place, which makes it difficult for the light emitting unit (35) and the light receiving unit (40) to slide or move. The material of which the arch wrap portion (8) is made is opaque to light, which effectively seals the pulse oximeter (30) from ambient outside light Even though the ankle wrap portion (7) may be tightly wrapped around the ankle of an infant, it will not affect the supply of arterial blood to the arch ball portion of the foot in which the pulse oximeter is mounted. Consequently, this design does as much as possible to eliminate artifactual readings from ambient light, low perfusion, or sensor movement.

Also shown in FIG. 2 is a warning light (23) that advises a user in the event the power source (25) becoming low on power. The charging inlet (24) is shown in proximity to the power source (25). Extending from the transmitter (20) in a direction opposite from the pulse oximeter (30) is a small wire antenna (13), which will ordinarily lie across the connecting portion (9) and extend up and into the ankle wrap portion (7). A very thin flexible wire antenna (13) is woven within the materials that are of the connecting portion (9) and the ankle wrap portion (7) without comprising either the elasticity or the comfort of the fit of the foot sensing unit (5). A variety of types of batteries may be employed as the power source (25). Two 9-volt mercury batteries are a common power source for pulse oximeters as they provide at least 8 hours of continuous operation for many pulse oximeter models. However, rechargeable nickel cadmium batteries are the preferred power source (25), but lithium batteries could also be used. The power source (25) must provide enough power for continuous operation of both the pulse oximeter (30) and the transmitting unit (20) for the period of time the foot sensing unit (5) would be in use. This would ordinarily constitute a normal sleep period for an infant, which would be 8 to 12 hours. The infant SIDS warning device (1) would not be employed continuously, because parents would have the infant under immediate observation in their presence for much of the day. When the infant SIDS warning device (1) is not in use, the power source (25) could be recharged or replaced as needed.

FIGS. 3 and 4 show the monitoring unit (70). In FIG. 3, a portion of the monitoring unit (70) is shown in cut-a-way for better visualization of the monitoring power source (82). The monitoring unit (70) is approximately the size of a cell phone receiver. Like a cell phone it will have a truncated antenna (76). The monitor power source (82) will ordinarily be a battery. Different batteries may function best for different applications, but ordinarily a rechargeable nickel cadmium battery is preferred. As with a cell phone receiver or a portable phone, the monitoring unit (70) may be connected directly to house current by means of a base recharging unit (100) (shown in FIG. 9). The base recharging unit (100) could serve to both recharge the monitor power source (82) and for operation of the monitoring unit (70) by household current where portable operation is not required. There is a sound generator (95) on the front of the monitoring unit (70). This gives an audible warning in the event an unacceptable low-blood oxygen reading is recorded. A warning light (93) advises the operator in the event the power source (82) is running low. This helps prevent malfunction of the device due to low power. At the top of the monitoring unit (70) is a read-out (90). This displays blood oxygen saturation readings (92) and a pulse rate (94). These are shown with typical values of 98% for blood oxygen saturation reading (92) and 100 for a pulse rate (94). This display will ordinarily be the standard liquid crystal display visible in most light conditions. FIG. 4 shows a middle portion of the monitoring unit (70) shown in cut-a-way. The monitoring power unit supply (82) (not shown) is connected to the controller unit (80) and to the receiver unit (75). The receiver unit (75) receives signals from the transmitting unit (20) in the foot sensing unit (5) (not shown). The receiving unit (75) translates the radio signals into an analog signal to the controller unit (80). The controller unit (80) has computer chips and logic circuitry required to translate the analog signal into a digital read-out (90), sowing both the blood oxygen saturation (92) and the pulse rate (94). The logic circuitry will activate the sound generator (95) in the event the logic circuitry determines the blood oxygen saturation (92) is too low for safety. The controller unit (80) can also sense when the monitoring power source (82) is low on power and activate the warning light (93).

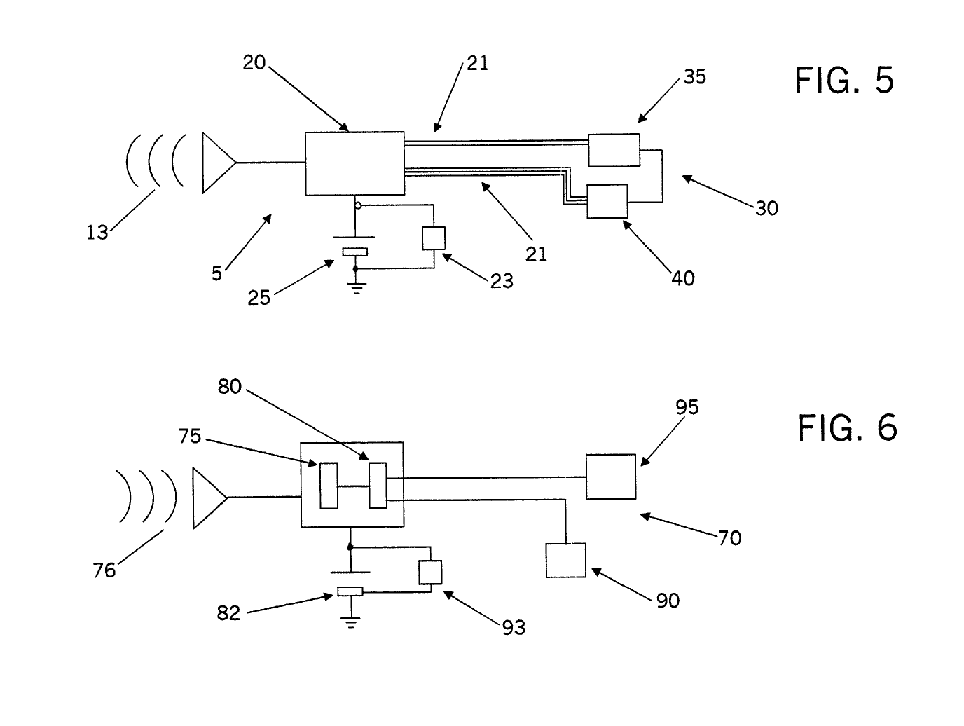

FIG. 5 shows in diagram form the foot sensing unit (5). The transmitting unit (20) is connected to the power source (25) and to an antenna (13). The power source (25) is wired through the transmitting unit through connecting wires (21) to the pulse oximeter (30). The light emitting unit (35) and the light sensing unit (40) are shown. A total of five different electrical connections connect the pulse oximeter (30) to the transmitting unit (20). Two of the electrical connections in the electrical connecting wires (21) take power to the light emitting unit (35). Two more electrical connections take power to the light sensing unit (40). One wire transmits the signals generated by the light sensing unit (40) to the transmitting unit (20). The transmitting unit (20), by means of the antenna (13), broadcasts a radio signal, shown as wavy lines emitting from the antenna (13), to the monitoring unit (70) (as seen in FIGS. 3 and 4). Also shown is a low-voltage sensor warning light (23). This advises the operator in the event the power source (25) is low. As an additional safeguard, in the event the transmitting unit (20) stops operating, the monitoring unit (70) will sound an alarm. Thus, if for some reason, the power supply (25) dies immediately or there is a short in the electrical circuit, even though the low-voltage warning light (23) may remain unlit, the monitoring unit (70) will still sound an alarm.

FIG. 6 shows the monitoring unit (70) in diagram form. There is an antenna (76) shown receiving radio signals generated by the foot sensing unit (5) (shown in FIGS. 1 and 2). These signals are transmitted to the receiving unit (75), which converts them into a signal sent to the controller unit (80). It is connected to the monitoring power supply (82). Also connected to the monitoring power supply (82) is the low battery warning light (93). The controller unit (80) generates a signal to the digital read-out (90) to generate O₂ and pulse readings. The controller unit (80) also sends signals to the sound generator (95) to sound an alarm if necessary.

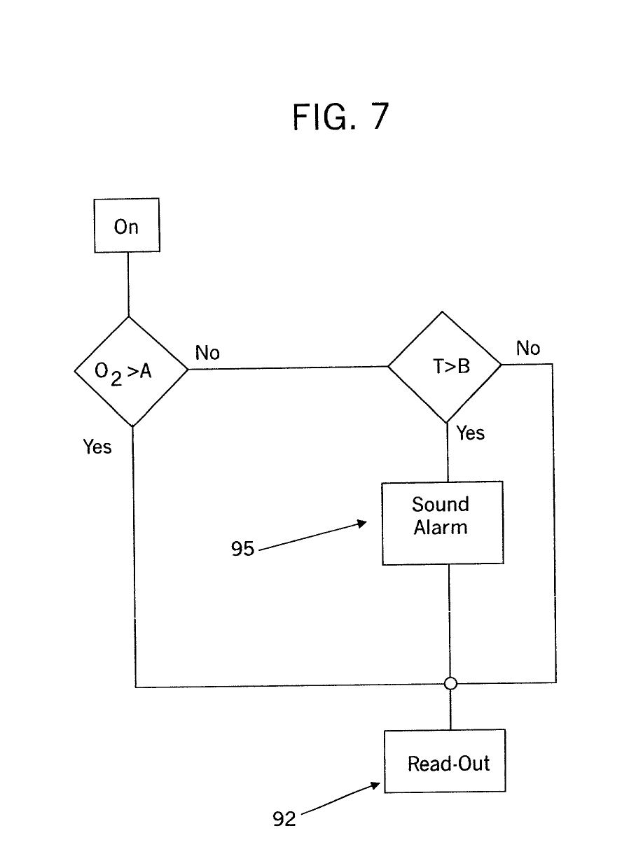

FIG. 7 shows the logic employed by the controller unit (80). Once the device is in place, there is a continuous check of the oxygen read-out. If the oxygen read-out is greater than a predetermined value of (A), then a signal is transmitted to the blood oxygen saturation read-out (92) to show the value of O₂. If the signal is below the predetermined value (A), then a signal goes to the time determinator. The O₂ values are continually sent from the pulse oximeter (30) by the transmitter (20). The first time a continuous reading drops below the predetermined value (A), this starts the time clock in the time determinator. As long as the O₂ value is below (A), the signal continues to feed into the time determinator. At the onset of the first low signal, the time clock starts running until a predetermined time (B) has elapsed. If, during this delay period (B) the O₂ read-out increases to where it is greater than the predetermined value (A), then the time stops and is re-set to zero and the read-out signal is again forwarded directly to the read-out (92). However, as long as the O₂ reading remains below (A), the signal is diverted to the time determinator. If, during the time (B) O₂ goes above (A), then no further action is taken. However, if the O₂ read-out remains below (A) for the predetermined time (B), then the alarm (95) sounds alerting the operator that the O₂ level has remained below the predetermined value (A) for the predetermined time (B).

The purpose of insuring there is a time delay between the first low O₂ below (A) and the sounding of the alarm is to avoid alarms caused by temporary low readings, which may be caused by the motion of the infant, unusual positions, or transient physical events like sneezing or coughing. Ordinarily, these predetermined values (A) and (B) will be set at the factory and cannot be changed by the operator. If the sound alarm (95) goes off, it alerts the parents their baby must be checked immediately to determine the cause of the low oxygen reading. If there is nothing apparently nothing wrong with the baby and it appears to be normal, the parents may turn off the monitoring unit (70) which re-sets the logic circuitry and stops the alarm (95). The monitoring unit (70) will be turned on, which starts the reception of the oxygen readings again. If continuous low oxygen readings continue to be received, the alarm will sound again. In this case, the parents may wish to check the foot sensing unit (5) to be sure it is still in position and that it is operating properly. If no signal is being received from the foot sensing unit (5), this will automatically cause the alarm (95) to go off, because no signal will be read by the logic circuitry as an O₂ reading of zero. Ordinarily, if the infant SIDS monitoring device (1) is functioning properly and the batteries are operating properly, then no more than a readjustment of the foot sensing unit (5) should be required for the operation to resume normal readings and for the alarm (95) to stop sounding. If the alarm (95) continues to sound after these precautionary measures are taken, it may be necessary to awaken the baby or to take other steps to be sure the low oxygen readings are artifactual, rather than a reflection of a serious respiratory distress on the part of the infant being monitor.

FIG. 8 shows the entire infant SIDS warning device (1) in place. The monitoring unit (70) is shown on a table by a chair having a parent or other caregiver in a living room. The foot sensing unit (5) is in place on the baby lying in a crib in a nursery. Wavy lines labeled “radio signals” are generated by the foot sensing unit (5) on the baby in the nursery and are received by the monitoring unit (70) in the living room where the caregiver is located. There is a continuous rad-out on the monitoring unit (70) of the blood oxygen saturation level and of the pulse rate of the baby to which the foot sensing unit (5) is affixed.

FIG. 9 shows the infant SIDS warning device (1) being recharged. The monitoring unit (70) is inserted into a recharging unit (100), which is connected to wall current by a standard electrical connecting cord (102). At the base of the monitoring unit (70) will be connections (101) for recharging the monitor power source (82) by current received from the wall plug (102) and passing through the appropriate transformer in the recharging unit (100). Likewise, there is a recharging cord (104), which connects to the charging inlet (24) on the foot sensing unit (5). When the foot sensing unit (5) is not in use and in place, it can be folded and placed in a convenient location next to the recharging unit (100). Ordinarily, when the infant SIDS warning device (1) is not in use, it will be placed in position (as shown in FIG. 9) for recharging. The indicator lights (105) on the recharging unit (100) indicate when both the monitoring unit (70) and the foot sensing unit (5) are recharging. When they are fully charged, the lights will go out. When the baby is awake and active, there is no reason for the infant SIDS warning device (1) to be in place or in use. It will be stored and recharged at its location on the recharging unit (100). Only when the baby is place din bed or otherwise in a position where there is vulnerability to a SIDS event will the foot sensing unit (5) be placed around the infants' foot and the monitoring unit (70) activated so the caregiver can give appropriate monitoring of the infant's condition.

I claim:

1. A portable adjustable cordless device to alert caregivers to the onset of a Sudden Infant Death Syndrome occurring in a premature, newly born, or very small infant comprising:
(a) a foot mounted apparatus wherein:
said apparatus has self-contained means for determining blood oxygen levels;
said apparatus has means for securing said means for determining in the plantar/arch area of an infant's foot so that said means for determining does not move forward and backward on an infant's foot or rotationally around an infant's foot;
said apparatus has self-contained cordless means for transmitting said blood oxygen levels determined by self-contained means for determining blood oxygen;
said apparatus has first means for powering said self-contained blood oxygen determining means and said self-contained transmitting means;
(b) a monitor to be kept with a caregiver wherein:
said monitor has means for receiving transmissions of blood oxygen determination from said cordless transmission means in said apparatus, said blood oxygen determination being made by said self-contained determining means in said apparatus;
said monitor has means for processing blood oxygen determinations;
said monitor has means for sounding an audible alarm at blood oxygen determinations fall below a predetermined level and;
said monitor has a second means for powering said receiving means, processing means, and said sounding means;
whereby a caregiver may secure the foot mounted apparatus around a foot of an infant and placing an infant so fitted with the foot mounted apparatus into a bed or other resting position and leaving an infant unattended but keeping said monitor with a caregiver so that a caregiver may listen for said audible alarm and so be alerted to an onset of Sudden Infant Death Syndrome.

2. A portable adjustable cordless device of claim 1 wherein said means for securing comprises an adjustable plantar/arch portion of the apparatus to wrap around a plantar/arch area of an infant's foot in a snug fashion and be secured against movement from a toe area of an infant's foot toward an ankle area of an infant's foot; an ankle wrap portion of the apparatus to wrap around an ankle of an infant's foot and connected to the plantar/arch wrap portion of the apparatus by a connecting portion so that the plantar/arch wrap portion of the apparatus is secured against motion from an ankle area of an infant's foot toward a toe area of an infant's foot and also is secured against a rotating motion around an infant's foot by the connecting portion secured to an ankle wrap portion of the apparatus, whereby said means for determining is fixedly mounted on an infant's foot in the plantar/arch area of an infant's foot and secured from forward or backward motion or rotational motion around an infant's foot.

3. A portable adjustable cordless device of claim 2 wherein said self-contained means for determining blood oxygen levels is a pulse oximeter mounted in the plantar/arch area of an infant's foot and secured within the adjustable portion of the apparatus that wraps around a plantar/arch area of an infant's foot.

4. A portable adjustable cordless device of claim 3 wherein said adjustable portion of the apparatus to wrap around the plantar/arch area of an infant's foot is constructed of opaque material, whereby ambient light does not result in artifactual readings of blood oxygen levels taken by said pulse oximeter.

5. A portable adjustable cordless device of claim 4 wherein said foot mounted apparatus is constructed, at least in part, of stretchy elasticized material.

6. A portable adjustable cordless device of claim 5 wherein for said pulse oximeter a light emitter is mounted on the top of an infant's foot and a light sensor is mounted on the bottom of the infant's foot in the plantar/arch area of an infant's foot.

7. A portable adjustable cordless device of claim 6 wherein said first means for powering and said second means for powering are batteries.

8. A portable adjustable cordless device of claim 7 wherein said monitor has means for displaying pulse readings generated by said pulse oximeter.

9. A portable adjustable cordless device of claim 8 wherein there is a first means for warning a caregiver wherein said first set of batteries is becoming depleted and a second means for warning a caregiver when said second set of batteries is becoming depleted.
